(19) United States
(12) Patent Application Publication
(10) Pub. No.: US 2024/0033005 A1
(43) Pub. Date: Feb. 1, 2024

(54) SYSTEMS AND METHODS FOR GENERATING VIRTUAL REALITY GUIDANCE

(71) Applicant: INTUITIVE SURGICAL OPERATIONS, INC., Sunnyvale, CA (US)

(72) Inventors: Prasad V. Upadrasta, San Jose, CA (US); Simon P. DiMaio, San Carlos, CA (US); Govinda Payyavula, Sunnyvale, CA (US); John Ryan Steger, Los Gatos, CA (US)

(21) Appl. No.: 18/255,336

(22) PCT Filed: Nov. 29, 2021
(86) PCT No.: PCT/US2021/060972

§ 371 Date: May 31, 2023

Related U.S. Application Data
(60) Provisional application No. 63/120,175, filed on Dec. 1, 2020.

(51) Int. Cl. A61B 34/10; A61B 34/20; A61B 34/35; A61B 90/00

(52) U.S. Cl. CPC A61B 34/10; A61B 34/20; A61B 34/35; A61B 90/361; A61B 2034/102; A61B 2034/105; A61B 2034/2055; A61B 2034/2065; A61B 2090/363; A61B 2090/365

(57) ABSTRACT

A system comprises a processor and a memory having computer readable instructions stored thereon. The computer readable instructions, when executed by the processor, cause the system to receive an image of a medical environment and identify a medical component in the image of the medical environment. The medical component may be disposed in a first configuration. The computer readable instructions, when executed by the processor also cause the system to receive kinematic information about the medical component and generate virtual guidance based on the kinematic information. The virtual guidance may include a virtual image of the medical component disposed in a second configuration.

### CROSS-REFERENCED APPLICATIONS

This application claims the benefit of U.S. Provisional Application 63/120,175 filed Dec. 1, 2020, which is incorporated by reference herein in its entirety.

This application incorporates by reference in their entireties U.S. Provisional Application No. 63/120,140, filed Dec. 1, 2020, titled “SYSTEMS AND METHODS FOR PLANNING A MEDICAL ENVIRONMENT” and U.S. Provisional Application No. 63/120,191, filed Dec. 1, 2020, titled “SYSTEMS AND METHODS FOR GENERATING AND EVALUATING A MEDICAL PROCEDURE.”

### FIELD

The present disclosure is directed to systems and methods for robot-assisted medical procedures and more specifically to identifying components in an image of a medical environment and using kinematic information about the identified components to generate guidance in the form of virtual reality images.

### BACKGROUND

The set-up, operation, trouble-shooting, maintenance, and storage of teleoperational robotic or robot-assisted systems often involves complex training and reference to training materials. Often, generic training instructions and training materials may be unable to anticipate the unique circumstances of a particular medical environment, including the dimensions of the operating space, the robot-assisted system equipment available in the environment, the peripheral equipment available in the environment, the location of utilities in the environment, the personnel in the environment, and other parameters associated with the robot-assisted system. Systems and methods are needed to assist medical personnel by providing virtual guidance that is customized to the components and constraints of the particular medical environment.

### SUMMARY

The embodiments of the invention are best summarized by the claims that follow the description.

Consistent with some embodiments, a system may comprise a processor and a memory having computer readable instructions stored thereon. The computer readable instructions, when executed by the processor, cause the system to receive an image of a medical environment and identify a medical component in the image of the medical environment. The medical component may be disposed in a first configuration. The computer readable instructions, when executed by the processor also cause the system to receive kinematic information about the medical component and generate virtual guidance based on the kinematic information. The virtual guidance may include a virtual image of the medical component disposed in a second configuration.

In some embodiments, a system may comprise a display system and a robot-assisted manipulator assembly configured for operating a medical instrument in a medical environment. The robot-assisted manipulator assembly may have a manipulator frame of reference. The system may also comprise a control system including a processing unit including one or more processors. The processing unit may be configured to receive an image of the medical environment and identify a medical component in the image of the medical environment. The medical component may be disposed in a first configuration. The processing unit may also be configured to receive kinematic information about the medical component and generate virtual guidance based on the kinematic information. The virtual guidance may include a virtual image of the medical component disposed in a second configuration.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a flowchart illustrating a method for generating virtual guidance according to some embodiments.

FIG. 2 is a schematic illustration of a robot-assisted medical system according to some embodiments.

FIG. 3 is an initial image of a medical environment according to some embodiments.

FIG. 4 is a guidance image of a medical environment according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

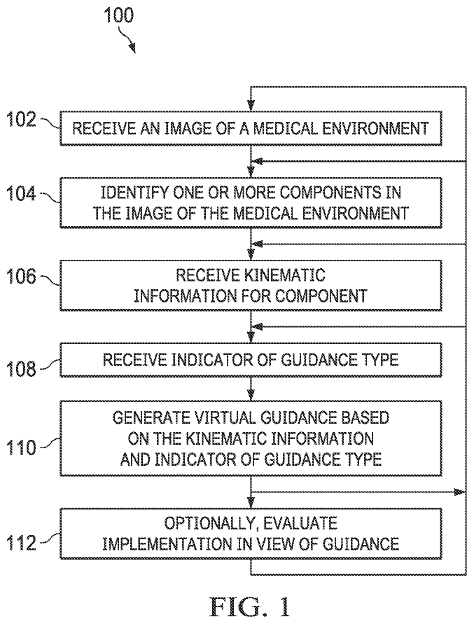

Guidance information may assist in the efficient, safe, and effective use of robot-assisted systems in a medical environment. As described below, guidance information that incorporates information about specific components in the medical environment may provide more detailed and customized guidance. FIG. 1 is a flowchart illustrating a method 100 for generating virtual guidance according to some embodiments. The methods described herein are illustrated as a set of operations or processes and are described with continuing reference to the additional figures. Not all of the illustrated processes may be performed in all embodiments of the methods. Additionally, one or more processes that are not expressly illustrated in may be included before, after, in between, or as part of the illustrated processes. In some embodiments, one or more of the processes may be implemented, at least in part, in the form of executable code stored on non-transitory, tangible, machine-readable media that when run by one or more processors (e.g., the processors of a control system) may cause the one or more processors to perform one or more of the processes. In one or more embodiments, the processes may be performed by a control system.

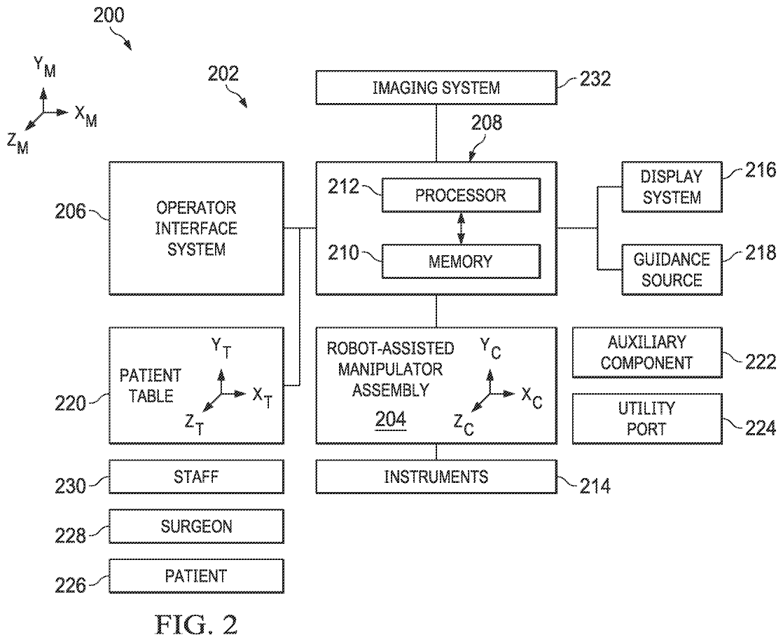

At a process 102, an image of a medical environment is received. FIG. 2 illustrates a medical environment 200 having a medical environment frame of reference (X_{M}, Y_{M}, Z_{M}) including a robot-assisted medical system 202 that may include components such as a robot-assisted manipulator assembly 204 having a component frame of reference (X_{C}, Y_{C}, Z_{C}), an operator interface system 206, and a control system 208. In one or more embodiments, the system 202 may be a robot-assisted medical system that is under the teleoperational control of a surgeon. In alternative embodiments, the medical system 202 may be under the partial control of a computer programmed to perform the medical procedure or sub-procedure. In still other alternative embodiments, the medical system 202 may be a fully automated medical system that is under the full control of a computer programmed to perform the medical procedure or sub-procedure with the medical system 202. One example of the medical system 202 that may be used to implement the systems and techniques described in this disclosure is the da Vinci® Surgical System manufactured by Intuitive Surgical Operations, Inc. of Sunnyvale, California. The medical environment 200 may be an operating room, a surgical suite, a medical procedure room, or other environment where medical procedures or medical training occurs.

The control system 208 may include at least one memory 210 and a processing unit including at least one processor 212 for effecting communication, control, and data transfer between components in the medical environment. Any of a wide variety of centralized or distributed data processing architectures may be employed in the control system 208. Similarly, the programmed instructions may be implemented as a number of separate programs or subroutines, or they may be integrated into a number of other aspects of the systems described herein, including teleoperational systems. In one embodiment, the control system 208 may support any of a variety of wired communication protocols or wireless communication protocols such as Bluetooth, IrDA, HomeRF, IEEE 802.11, DECT, and Wireless Telemetry. In some embodiments, the control system 208 may be in a different environment, partially or entirely remote from the manipulator assembly 204 and the operator interface system 206, including a different area of common surgical environment, a different room, or a different building.

The manipulator assembly 204 may be referred to as a patient side cart. One or more medical instruments 214 (also referred to as a tools) may be operably coupled to the manipulator assembly 204. The medical instruments 214 may include end effectors having a single working member such as a scalpel, a blunt blade, a needle, an imaging sensor, an optical fiber, an electrode, etc. Other end effectors may include multiple working members, and examples include forceps, graspers, scissors, clip appliers, staplers, bipolar electrocautery instruments, etc. The number of medical instrument 214 used at one time will generally depend on the medical procedure and the space constraints within the operating room among other factors. A medical instrument 214 may also include an imaging device. The imaging instrument may comprise an endoscopic imaging system using optical imaging technology or comprise another type of imaging system using other technology (e.g. ultrasonic, fluoroscopic, etc.). The manipulator assembly 204 may include a kinematic structure of one or more links coupled by one or more non-servo controlled joints, and a servo-controlled robotic manipulator. In various implementations, the non-servo controlled joints can be manually positioned or locked, to allow or inhibit relative motion between the links physically coupled to the non-servo controlled joints. The manipulator assembly 204 may include a plurality of motors that drive inputs on the medical instruments 214. These motors may move in response to commands from the control system 208. The motors may include drive systems which when coupled to the medical instrument 214 may advance the medical instrument into a naturally or surgically created anatomical orifice in a patient. Other motorized drive systems may move the distal end of the medical instrument in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the motors can be used to actuate an articulable end effector of the instrument for grasping tissue in the jaws of a biopsy device or the like. Kinematic information about the manipulator assembly 204 and/or the instruments 214 may include structural information such as the dimensions of the components of the manipulator assembly and/or medical instruments, joint arrangement, component position information, component orientation information, and/or port placements. Kinematic information may also include dynamic kinematic information such as the range of motion of joints in the teleoperational assembly, velocity or acceleration information, and/or resistive forces. The structural or dynamic kinematic constraint information may be generated by sensors in the teleoperational assembly that measure, for example, manipulator arm configuration, medical instrument configuration, joint configuration, component displacement, component velocity, and/or component acceleration. Sensors may include position sensors such as electromagnetic (EM) sensors, shape sensors such as fiber optic sensors, and/or actuator position sensors such as resolvers, encoders, and potentiometers.

The operator interface system 206 allows an operator such as a surgeon or other type of clinician to view images of or representing the procedure site and to control the operation of the medical instruments 214. In some embodiments, the operator interface system 206 may be located in the same room as a patient during a surgical procedure. However, in other embodiments, the operator interface system 206 may be located in a different room or a completely different building from the patient. The operator interface system 206 may generally include one or more control device(s) for controlling the medical instruments 214. The control device(s) may include one or more of any number of a variety of input devices, such as hand grips, joysticks, trackballs, data gloves, trigger-guns, foot pedals, hand-operated controllers, voice recognition devices, touch screens, body motion or presence sensors, and the like. In some embodiments, the control device(s) will be provided with the same degrees of freedom as the medical tools of the robotic assembly to provide the operator with telepresence; that is, the operator is provided with the perception that the control device(s) are integral with the tools so that the operator has a sense of directly controlling tools as if present at the procedure site. In other embodiments, the control device(s) may have more or fewer degrees of freedom than the associated medical tools and still provide the operator with telepresence. In some embodiments, the control device(s) are manual input devices which move with six degrees of freedom, and which may also include an actuatable handle for actuating medical tools (for example, for closing grasping jaw end effectors, applying an electrical potential to an electrode, capture images, delivering a medicinal treatment, and the like). The manipulator assembly 204 may support and manipulate the medical instrument 214 while an operator views the procedure site through a display on the operator interface system 206. An image of the procedure site can be obtained by the imaging instrument, such as a monoscopic or stereoscopic endoscope, which can be manipulated by the manipulator assembly 204.

Another component that may, optionally, be arranged in the medical environment 200 is a display system 216 that may be communicatively coupled to the control system 208. The display system 216 may display, for example, images, instructions, and data for conducting a robot-assisted procedure. Information presented on the display system 216 may include endoscopic images from within a patient anatomy, guidance information, patient information, and procedure planning information. In some embodiments, the display system may be supported by an electronics cart that allows for mobile positioning of the display system.

A guidance source 218 may be communicatively coupled to the control system 208 or may be stored in the memory 210. The guidance source 218 may include stored information including best practice information and historical procedure information. For example, the guidance source may include sample medical environment layouts for various procedures. Additionally or alternatively the guidance source 218 may include personnel including experts, trainers, mentors, or other guidance staff that may support a user experience. The guidance source 218 may, optionally, be located outside of the medical environment 200.

Other medical components in the medical environment 200 that may or may not be communicatively coupled to the control system 208 may include a patient table 220, which may have a table frame of reference (X_{T}, Y_{T}, Z_{T}), and an auxiliary component 222 such as an instrument table, an instrument basin, an anesthesia cart, a supply cart, a cabinet, and seating. Other components in the medical environment 200 that may or may not be communicatively coupled to the control system 208 may include utility ports 224 such as electrical, water, and pressurized air outlets.

People in the medical environment 200 may include the patient 226 who may be positioned on the patient table 220, a surgeon 228 who may access the operator interface system 206, and staff members 230 which may include, for example surgical staff or maintenance staff.

Referring again to FIG. 1, at the process 102, an image of the medical environment may be received from an imaging system 232. The imaging system 232 may be a camera or other imaging device located in or capable of recording an image in the medical environment 200. In some embodiments, the imaging system 232 may be a portable camera including, for example, a camera incorporated into a mobile phone, a tablet, a laptop computer, or other portable device supported by a surgeon 228 or staff member 230. Additionally or alternatively, the imaging system 232 may include a camera or a plurality of cameras mounted to the walls, floor, ceiling, or other components in the medical environment 200 and configured to capture images of the components and personnel within the medical environment. In some embodiments, the imaging system may include other types of imaging sensors including, for example, a lidar imaging system that may scan the environment to generate three-dimensional images using reflected laser light. In some embodiments, the captured image may be a composite image generated from multiple images. In some embodiments, the received image may be a two-dimensional or a three-dimensional image.

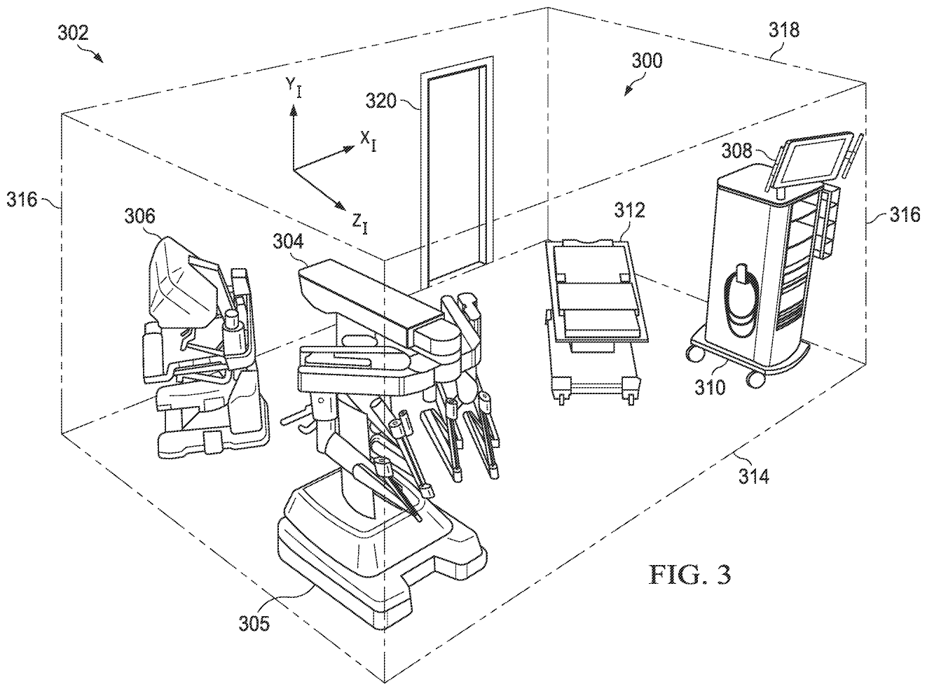

FIG. 3 is an initial image 302 of a medical environment 300 that may be received at process 102. The image 302 may be three-dimensional and may be generated with lidar technology or with composite images from a mobile phone camera. The image 302 may include an image of movable components including a manipulator assembly 304 (e.g., the manipulator assembly 204) with a base 305, an operator interface system 306 (e.g., the operator interface system 206), a display 308, a cart 310, and a patient table 312. The image 302 may also include stationary components including the floor 314, walls 316, ceiling 318, and door 320. The dimensions of the room 300 may be determined from the initial image 302. The image 302 may have an image frame of reference (X_{I}, Y_{I}, Z_{I}).

Referring again to FIG. 1, at a process 104, one or more components are identified in the image of the medical environment. For example, in the image 302 of medical environment 300, a manipulator assembly 304 may be identified using image recognition software that recognizes a component or a portion of a component in an image based on shape, color, fiducial markings, alphanumeric coding, or other visually identifiable characteristics. Alternatively, a user may provide an indication of an identified component in the image. The pixels or voxels associated with the identified component(s) may be graphically segmented from the image. In the image 302, image recognition software may identify the base 305 of the manipulator assembly 304 and may associate the recognized base 305 with a specific model of the manipulator assembly 304. Similarly, the recognized component may be the operator interface system 306, the patient table 312, the cart 310, and/or the display 308.

The image frame of reference may be registered to the identified component frame of reference. For example, the image 302 frame of reference (X_{I}, Y_{I}, Z_{I}) may be registered to the manipulator assembly 304 frame of reference (e.g. frame of reference (X_{C}, Y_{C}, Z_{C})). Common or fiducial features or portions may be identified and matched (e.g. in position and/or orientation) in both the image frame of reference and the component frame of reference to perform the registration. Such fiducial features or portions may include the manipulator base, the manipulator column, the manipulator boom, and/or manipulator arms. Three-dimensional images or two-dimensional images from different vantage points may provide a more accurate registration. With the image frame of reference registered to the manipulator frame of reference, the position and orientation of the manipulator arms, joints, and attached instruments may be determined in the image frame of reference. Thus, any virtual motions of the manipulator assembly, including the corresponding changes in arm, joint, or instrument position/orientation, that are possible based on the manipulator assembly kinematics may be rendered virtually in the image frame of reference. Alternatively or additionally, the image frame of reference may be registered to the medical environment frame of reference (X_{M}, Y_{M}, Z_{M}) or to the frames of reference of other components visible in the image such as the patient table frame of reference (X_{T}, Y_{T}, Z_{T}).

At a process 106, kinematic information for the identified component may be received. For example, kinematic information about the manipulator assembly 304 and/or any coupled instruments (e.g. instruments 214) may include structural information such as the dimensions of the components of the manipulator assembly and/or medical instruments, joint arrangement, component position information, component orientation information, and/or port placements. Kinematic information may also include dynamic kinematic information such as the range of motion of joints in the teleoperational assembly, velocity or acceleration information, and/or resistive forces. The structural or dynamic kinematic constraint information may be generated by sensors in the teleoperational assembly that measure, for example, manipulator arm configuration, medical instrument configuration, joint configuration, component displacement, component velocity, and/or component acceleration. Sensors may include position sensors such as electromagnetic (EM) sensors, shape sensors such as fiber optic sensors, and/or actuator position sensors such as resolvers, encoders, and potentiometers.

At a process 108, a guidance type indicator is received. The guidance type indicator may be an indication of the type of guidance needed by the user or needed by the medical system to perform a new process. The indicator may, for example, be received at the control system 208 from a mobile device including the imaging system 232, the operator interface system 206, the display system 216, the manipulator assembly 204 or other component in communication with the control system 208. In some embodiments, the guidance type indicator may include an indicator of a mode of operation for the identified component. Modes of operation that may be indicated include a set-up mode for preparing the manipulator assembly and the medical environment to begin a medical procedure. The set-up mode may include a sterile preparation mode in which sterile and non-sterile areas of the medical environment are defined. The sterile and non-sterile areas may be any two or three-dimensional areas within the medical environment. In the sterile preparation mode, the manipulator assembly may be arranged to receive a sterile drape, and the draping may be arranged over the manipulator assembly. In some embodiments, the draping procedure may include multiple choreographed steps. The modes of operation may also include a procedure mode in which the draped manipulator assembly is prepared to perform a medical procedure. Other modes of operation may include an instrument exchange mode in which an instrument coupled to the manipulator assembly is exchanged; a trouble-shooting mode in which the mid-procedure manipulator assembly requires attention by an operator to change an instrument or correct a performance issue with the manipulator assembly; and a servicing mode in which the manipulator assembly receives routine maintenance or repair service. Other modes of operation may include an inspection mode in which manipulator is inspected for damage and compliance to manufacturer's standards; a cleaning mode in which the manipulator assembly is disinfected, sterilized, or otherwise cleaned; and a storage mode in which the manipulator assembly is stowed before and after a medical procedure or otherwise out of use.

At a process 110, virtual guidance may be generated based on the inputs of the kinematic information and the guidance type indicator. The virtual guidance may include static or dynamic/animated images and may include two-dimensional or three-dimensional images. The virtual guidance may, for example, include a virtual image (e.g., an artificially generated image) of the component moved to a new position in the medical environment or arranged in a different configuration in the medical environment. Generating virtual guidance may include referencing guidance information from the guidance source 218 which may include stored user training information, prior procedure information, best practice information, reference models of the component in a variety of operation modes, a mentor-approved procedure, or expert practice information. The guidance information may be combined with the kinematic information to generate artificial still images or animations that demonstrate how to set-up the component, perform a task using the component, trouble-shoot an operational issue with the component, repair the component, or stow the component when out of use.

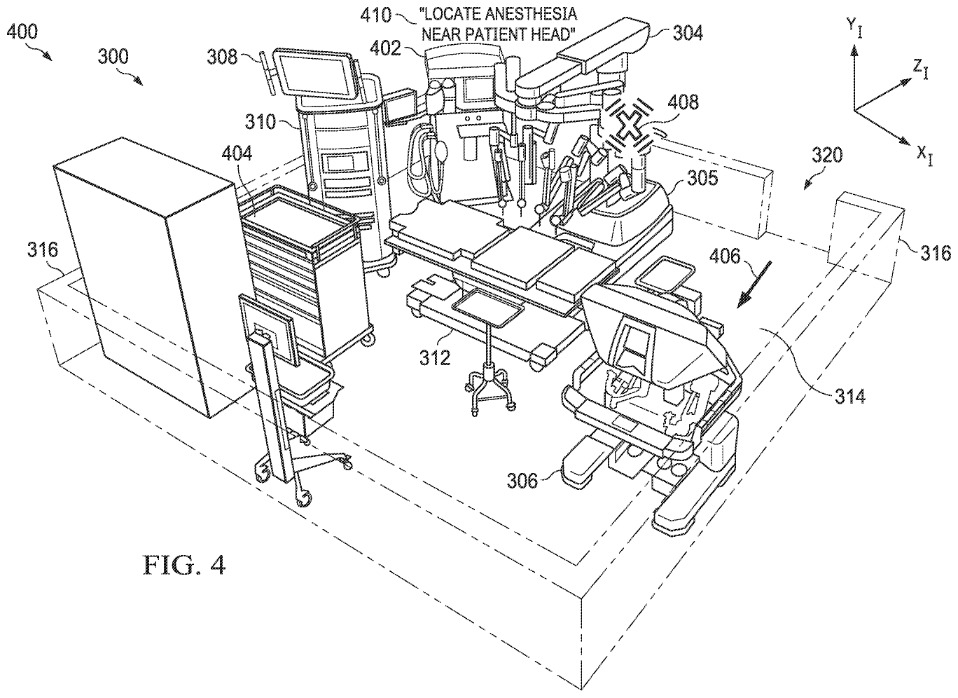

As an example, the virtual guidance may be a virtual animation or image that demonstrates how the identified components in the medical environment 300 may be arranged to perform a procedure. The virtual guidance may illustrate how to move components within the medical environment 300 and/or how to introduce components into the medical environment. FIG. 4 illustrates a virtual guidance image 400 of the medical environment 300 arranged to perform a procedure. Based on the kinematic information and the guidance information, the virtual guidance image 400 renders illustrations of the manipulator assembly 304, the patient table 312, the operator interface system 306, the display 308, and the cart 310 in a new position and configuration suitable for performing the procedure. The virtual guidance image 400 also includes other suggested components such as an anesthesia cart 402 and an instrument cart 404 and the preferred positions for the suggested components. Known kinematic information about the components, including size and range of motion may inform surrounding clearance areas, access areas, paths for staff travel, and other constraints on the component layout. In some examples, the transition between the initial image 302 and the virtual guidance image 400 may be animated with movements in the animation constrained by the known kinematics for the identified components. The virtual guidance may also include renderings of virtual staff members, the surgeon, and/or the patient, including, for example, traffic routes, sterile areas, access paths, personalized instructions or other guidance for personnel placement or movement. The virtual guidance image 400 may include annotations or graphical markers such as symbols, color indicators, animated indicators to provide additional guidance. For example, directional indicators 406 may be used to indicate a travel path or direction for component movement. Attention indicators 408 may be symbols that may be animated (e.g. flashing, shaking) and/or brightly or unusually colored to attract a viewer's attention. Because the component images themselves are all virtually rendered, the component or a portion of the component may be animated or rendered in an artificial color to attract a viewer's attention. Annotations 410 may also be provided to provide additional information or instruction. In some embodiments, a guidance animation may demonstrate how to arrange the manipulator assembly 304 into a stowage configuration or into a draping configuration. In some embodiments, a guidance animation may demonstrate procedure steps such as how to perform an instrument exchange procedure in which a first instrument is removed from the manipulator assembly 304 and is replaced with a second instrument or how to establish proper anatomical port placements. In some embodiments, the guidance animation may demonstrate how to perform a corrective action to correct, for example, and improperly installed instrument, manipulator arms incorrectly positioned at the start of a procedure, or arm positions that will or have resulted in collision.

In other examples, the virtual guidance may be delivered during a procedure. The guidance indicator may be, for example, a malfunctioning tool or a manipulator arm collision that prompts the generation of virtual guidance. Based on kinematic information received from the manipulator assembly, the virtual guidance may include virtually rendered flashing symbols or highlighted component parts that indicate required attention, such as a malfunctioning instrument or collided arms.

In some embodiments, the virtual guidance may be displayed. For example, the still or animated virtual guidance images may be displayed on the mobile device comprising the imaging system 232 that was used to generate the original image, the operator interface system 206, or the display system 216. In some embodiments, the virtual guidance may be displayed with or may be superimposed or overlayed on the initial image. In some embodiments, the virtual guidance may be displayed on a display of the operator interface system and/or on one or more auxiliary display devices in the medical environment. In some embodiments, the virtual guidance may be conveyed using another sensory system such as an auditory system that generates audible guidance.

Optionally, after the virtual guidance is generated, any or all of the processes 102-110 may be repeated. For example, after virtual guidance is generated for a guidance type that corresponds to a set-up mode of operation, the processes 108 and 110 may be repeated for a deployment or procedural mode of operation to generate guidance to conduct the procedure deploying the manipulator assembly.

At a process 112 that may be optional, an implementation of the virtual guidance is evaluated. The implementation may be evaluated based on a comparison to the virtual guidance. For example, after the medical environment 300 is arranged in preparation for a procedure, an evaluation may be performed to determine whether or to what extent the real arrangement of the components in the medical environment 300 matches the virtual guidance. The evaluation may be based on kinematic information received from the arranged components, including for example the manipulator assembly 204, and/or images received from the imaging system 232 after the components are arranged.

In some embodiments, the method 100 may be used in a practice or training scenario for education of clinical staff or surgeons. In a training scenario, the virtual guidance may be displayed on one or more display devices, including one or more mobile devices, a surgeon console, and/or a mobile or stationary auxiliary display in the medical environment. The training scenario may be a program component of a curriculum, and the process 112 may include providing evaluation data such as feedback to the clinical staff or surgeons from a remote mentor and/or a score or grade based upon the evaluation of the implemented plan compared to the virtual guidance. In some embodiments, the evaluation data may be displayed to the clinical staff or surgeons. In other embodiments, the evaluation data may not be displayed to the clinical staff or surgeons but may be provided to a proctor, mentor, curriculum development organization, medical system manufacturer, or other individual or organization that may use the evaluation data for other purposes such as system evaluation or procedure improvement. The evaluation data may be used to provide warnings, suggestions, or assistance during subsequent procedures with the clinical staff or surgeons.

Optionally, after the evaluation, any or all of the processes 102-112 may be repeated. For example, after a set-up procedure is implemented and evaluated based on a comparison to the guidance, a determination may be made that the set-up procedure was not successful or was not performed in accordance with the virtual guidance. The processes 102-110 may be repeated with a new image of the medical environment with the components in their current state and with guidance type that corresponds to a set-up mode of operation. Thus, new virtual guidance may be generated to correct the set-up.

In some embodiments, the kinematic information received at process 106 may be used to identify a stored reference model of the component. The reference model may be registered to the component. For example, the memory 210 may store a plurality of models of a manipulator assembly. The models may include various models of a manipulator assembly and various mode configurations for each model. For example, static or dynamic models may be stored for a stowed configuration, a deployed configuration, a draping configuration, a patient positioning configuration, a tool change configuration, or any other configuration associated with a mode of operation of the manipulator assembly. The received kinematic information may be compared to or matched with the stored models to select a reference model for the current configuration of the manipulator assembly. In some embodiments, the selected reference models may be adjusted based on the actual received kinematic information. The models may be used generate the virtual guidance at the process 110. While the guidance is implemented, the model may be registered to the component and may be dynamically updated based on the movement of the component.

Elements described in detail with reference to one embodiment, implementation, or application optionally may be included, whenever practical, in other embodiments, implementations, or applications in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

Any alterations and further modifications to the described devices, systems, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately.

Various systems and portions of systems have been described in terms of their state in three-dimensional space. As used herein, the term “position” refers to the location of an object or a portion of an object in a three-dimensional space (e.g., three degrees of translational freedom along Cartesian X, Y, Z coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom).

Although some of the examples described herein refer to surgical procedures or instruments, or medical procedures and medical instruments, the techniques disclosed optionally apply to non-medical procedures and non-medical instruments. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

A computer is a machine that follows programmed instructions to perform mathematical or logical functions on input information to produce processed output information. A computer includes a logic unit that performs the mathematical or logical functions, and memory that stores the programmed instructions, the input information, and the output information. The term “computer” and similar terms, such as “processor” or “controller” or “control system,” are analogous.

While certain exemplary embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

1. A system comprising:
a processor; and
a memory having computer readable instructions stored thereon, the computer readable instructions, when executed by the processor, cause the system to:
receive an image of a medical environment;
identify a medical component in the image of the medical environment, the medical component disposed in a first configuration;
receive kinematic information about the medical component; and
generate virtual guidance based on the kinematic information, the virtual guidance including a virtual image of the medical component disposed in a second configuration.

2. The system of claim 1 wherein the computer readable instructions, when executed by the processor, further cause the system to:
receive an indicator of guidance type.

3. The system of claim 1 wherein the computer readable instructions, when executed by the processor, further cause the system to:
provide an evaluation of an implementation compared to the virtual guidance.

4. The system of claim 1 wherein the medical component is a robot-assisted manipulator assembly.

5. The system of claim 1 wherein receiving the image includes receiving the image from a mobile device.

6. The system of claim 1 wherein receiving the image includes receiving the image from a camera system mounted in the medical environment.

7. The system of claim 1 wherein the image has an image frame of reference and the medical component has a component frame of reference and wherein the computer readable instructions, when executed by the processor, further cause the system to register the image frame of reference to the component frame of reference.

8. The system of claim 7 wherein registering the image frame of reference to the component frame of reference includes identifying a fiducial portion of the medical component in both the image frame of reference and the component frame of reference.

9. The system of claim 7, wherein the computer readable instructions, when executed by the processor, further cause the system to display the virtual image in the image frame of reference.

10. The system of claim 1 wherein receiving kinematic information about the medical component includes receiving sensor information from the medical component.

11. The system of claim 1 wherein the second configuration is a stowage configuration and the virtual image includes a virtual animation of the medical component being arranged in the stowage configuration.

12. The system of claim 1 wherein the second configuration is a draping configuration and the virtual image includes a virtual animation of the medical component being arranged in the draping configuration.

13. The system of claim 1 wherein the virtual image includes a virtual animation of a procedure step.

14. The system of claim 1 wherein the virtual image includes a virtual image of an auxiliary component.

15. The system of claim 14 wherein the virtual image includes a virtual animation of a set-up procedure for the medical component and the auxiliary component.

16. The system of claim 1 wherein the virtual image includes a virtual image of a patient
wherein the virtual image includes a virtual animation including the patient and the medical component.

17. The system of claim 1 wherein displaying the virtual image includes overlaying the virtual image on an image of the medical component in the first configuration.

18-35. (canceled)

36. The system of claim 1 further comprising a display system configured to display the virtual image.

37. The system of claim 1 further comprising a robot-assisted manipulator assembly configured for operating a medical instrument in the medical environment.

38. The system of claim 37 wherein the image has an image frame of reference registered to a manipulator frame of reference of the robot-assisted manipulator assembly.
